(19) United States
(12) Patent Application Publication
(10) Pub. No.: US 2026/0243762 A1
(43) Pub. Date: Aug. 20, 2026

(54) MULTIMODAL LATERAL FLOW ASSAY SYSTEM FOR SENSITIVE AND QUANTITATIVE DETECTION OF INFLAMMATION MARKERS

(71) Applicant: City University of Hong Kong, Hong Kong (HK)

(72) Inventors: Bee Luan KHOO, Hong Kong (HK); Wei LI, Hong Kong (HK); Xinrui WANG, Hong Kong (HK)

(21) Appl. No.: 19/435,790

(22) Filed: Dec. 30, 2025

Related U.S. Application Data
(60) Provisional application No. 63/759,514, filed on Feb. 17, 2025.

(51) Int. Cl. G01N 33/543; G01N 21/65; G01N 33/553

(52) U.S. Cl. CPC G01N 33/54388; G01N 21/658; G01N 33/553; G01N 2446/86

(57) ABSTRACT

A multimodal lateral flow assay (LFA) system for non-invasive biomarker monitoring is provided. The system includes a multimodal LFA strip having a sample-loading region for receiving a biological sample, a conjugate region containing triple-mode probes, and a membrane zone with immobilized capture and secondary antibodies that form respective test and control lines upon binding the probes. The triple-mode probes generate colorimetric, fluorescence, and surface-enhanced Raman scattering (SERS) signals. A laser-emitting module illuminates the membrane zone to excite fluorescence and SERS responses, which are detected by one or more optical detection devices. A processor performs multimodal signal mapping by analyzing fluorescence and SERS outputs to determine the quantitative concentration of the biomarker in the sample. The system enables sensitive, quantitative, and non-invasive detection of biomarkers across multiple optical modalities.

### CROSS-REFERENCE TO RELATED APPLICATIONS

The present application claims priority from U.S. Provisional Utility Patent application No. 63/759,514 filed Feb. 17, 2025; the disclosure of which is incorporated herein by reference in its entirety.

### FIELD OF THE INVENTION

The present invention generally relates to the field of bioanalytical sensing technology. More specifically, the present invention relates to multimodal lateral flow assay systems and methods for non-invasive, point-of-care detection and quantification of biomarkers.

### BACKGROUND OF THE INVENTION

Inflammation is a fundamental immune response triggered by infection, injury, or other pathological stimuli, and it plays a central role in numerous diseases, including atherosclerosis, Alzheimer's disease, rheumatoid arthritis, and various types of cancer. Biomarkers of inflammation, particularly C-reactive protein (CRP), are widely used to assess disease activity and inform therapeutic decisions. Compared with blood-based assays, urinalysis offers a promising non-invasive alternative for disease monitoring due to its ease of collection and suitability for frequent sampling. Urinary CRP has been correlated with several disease states; however, because its concentration is usually very low in healthy individuals and can vary substantially during different stages of inflammation, highly sensitive detection methods with a broad dynamic range are required for accurate quantification. A comprehensive understanding of inflammatory status through precise biomarker measurement is therefore essential for enabling the effective modulation of immune responses.

The widespread adoption of lateral flow assays (LFAs) during the COVID-19 pandemic has demonstrated their utility for large-scale testing in both clinical and decentralized settings. LFAs are valued for their rapid signal readout, portability, and user-friendly format, which allows for self-testing. Nonetheless, conventional gold nanoparticle (AuNP)-based LFAs suffer from limited sensitivity and a narrow dynamic range, which restricts their applicability for biomarkers present at low or variable concentrations. To address these limitations, various strategies—such as improving assay kinetics, implementing signal amplification schemes, and employing sample preamplification—have been developed to achieve analytical performance approaching that of enzyme-linked immunosorbent assays (ELISA) and polymerase chain reaction (PCR). Furthermore, the integration of nanomaterials as signal transducers or receptor-immobilization platforms has significantly enhanced the quantitative capabilities of LFAs.

A range of advanced LFA modalities employing optical, thermal, magnetic, and electrochemical detection principles has emerged to further enhance sensitivity and dynamic range, with multiple efforts progressing toward commercialization. Beyond traditional AuNP-based colorimetric formats, fluorescence-based LFAs and surface-enhanced Raman scattering (SERS) LFAs have demonstrated markedly higher sensitivity, enabling detection of biomarkers at much lower concentrations.

In view of these developments, the present invention seeks to provide a multimodal lateral flow assay system that uniquely integrates colorimetric, fluorescence, and SERS detection within a single platform for quantitative biomarker analysis across an extensive dynamic range. The multimodal assay enables highly sensitive detection of clinically relevant biomarkers, including those present at low concentrations in urine, and supports long-term, non-invasive monitoring of inflammatory status. This integrated LFA format offers significant potential as an in vitro diagnostic tool for managing chronic diseases and monitoring longitudinal health.

### SUMMARY OF THE INVENTION

The objective of the present invention is to provide systems and methods that solve the aforementioned technical problems.

In accordance with a first aspect of the present invention, a multimodal LFA system is provided for non-invasively monitoring biomarkers. The system includes a multimodal LFA stripe. The multimodal LFA stripe includes: a sample-loading region configured to receive a biology sample comprising a biomarker; a conjugate region comprising triple-mode probes; and a membrane zone immobilized with a capture antibody configured to bind complexes of the triple-mode probes and the biomarker and form a test line, and a secondary antibody configured to bind the triple-mode probes and create a control line, wherein the triple-mode probes are operable to generate a colorimetric signal. The conjugate region is disposed adjacent to the sample-loading region, and the membrane zone is disposed adjacent to the conjugate region. The system also includes: a laser-emitting module configured to illuminate the membrane zone to generate a surface-enhanced Raman scattering (SERS) signal and a fluorescence signal from the triple-mode probes; one or more optical detection devices configured to detect and receive the SERS signal and the fluorescence signal; and a processor configured to execute a multimodal mapping for analyzing the SERS signal and the fluorescence signal to obtain a concentration of the biomarker.

In accordance with one embodiment, the triple-mode probes includes: a metal-chelate polymer nanoparticle having carboxyl groups on its surface and configured to emit the fluorescence signal; a gold nanoparticle functionalized with binders and 4-nitrobenzenethiol (pNTP) configured to produce the colorimetric signal and the SERS signal under laser excitation; and an antibody capable of binding both the biomarker in the biological sample and the secondary antibody in the membrane zone. Specifically, the binders bind to the carboxyl groups, and the antibody is coupled to the metal-chelate polymer nanoparticle via the carboxyl groups.

In accordance with another embodiment, the multimodal mapping includes: extracting fluorescence and SERS intensity ratios from the test and control lines; applying a predetermined multimodal mapping coefficient to convert the fluorescence and SERS ratios into a unified response value; calibrating the unified response value by subtracting a blank-sample response; and determining the biomarker concentration in the biological sample based on a calibrated curve correlating standard concentrations with calibrated response values.

In accordance with yet another embodiment, the metal-chelate polymer nanoparticle includes a europium-chelate-doped polystyrene nanoparticle.

In accordance with yet another embodiment, the binder includes a polyethylene glycol (PEG) linker comprising thiol and amino functional groups.

In accordance with yet another embodiment, the multimodal LFA system further includes an absorbent region positioned downstream of the membrane zone for absorbing excess biological sample.

In accordance with yet another embodiment, the multimodal LFA system further includes a backing layer providing mechanical support and structural integration to the multimodal LFA stripe.

In accordance with yet another embodiment, the multimodal mapping coefficient is derived from standard samples by correlating fluorescence intensity ratios (R_{F}) and SERS intensity ratios (R_{R}) with known biomarker concentrations.

In accordance with yet another embodiment, the processor determines the biomarker concentration by applying the calibrated response value to a calibration curve.

In accordance with yet another embodiment, the processor controls exposure duration, laser intensity, and spatial positioning of the laser-emitting module to optimize SERS signal acquisition.

In accordance with yet another embodiment, the colorimetric signal is acquired using a camera integrated into a smartphone, tablet, or portable imaging module operatively connected to the processor.

In accordance with a second aspect of the present invention, a method for detecting or quantifying a biomarker in a biological sample using the aforementioned multimodal LFA system. The method includes the following steps: applying a biological sample to the sample-loading region of the multimodal LFA stripe; allowing the sample to migrate along the stripe through the conjugate region comprising the triple-mode configured to generate the complexes of the triple-mode probes and the biomarker, to create colorimetric, fluorescence, and Raman scattering signals; capturing the complexes of the triple-mode probe at the test line of the membrane zone; capturing excess multimodal probes at the control line of the membrane zone; acquiring multimodal optical signals from the test line and the control line using the one or more optical detection devices; and processing the optical signals by the processor to generate a quantitative measurement of the biomarker concentration. The processing step involves applying a multimodal mapping technique that combines colorimetric, fluorescence, and SERS signals.

In accordance with one embodiment, the steps of generating the quantitative biomarker measurement include computing signal ratios between the test line and the control line.

In accordance with another embodiment, the biological sample includes urine, saliva, tears, blood, serum, plasma, and cell culture media.

In accordance with yet another embodiment, the biomarker concentration is displayed to a user on a smartphone, tablet, or portable optical reader operatively connected to the processor.

In accordance with yet another embodiment, the fluorescence, colorimetric, and Raman signals are acquired sequentially or simultaneously.

In accordance with yet another embodiment, the processing further includes a step of normalizing fluorescence and Raman intensity ratios using control-line signals to reduce strip-to-strip variability.

### BRIEF DESCRIPTION OF THE DRAWINGS

Embodiments of the invention are described in more details hereinafter with reference to the drawings, in which:

FIGS. 1A-1E depict the development and performance of a multimodal lateral flow assay system according to one embodiment of the present invention, in which: FIG. 1A shows a flow chart of analyzing samples using the multimodal lateral flow assay system; FIG. 1B depicts a schematic of the multimodal lateral flow assay system, including the sample pad for applying samples, the conjugation pad for CFSERS probe retention, the nitrocellulose membrane for control and test line detection, and the absorbance pad for waste collection; FIG. 1C depicts the representative colorimetric results from the multimodal lateral flow assay system showing a positive result with both a control and test line and a negative result with only the control line; FIG. 1D depicts the fluorescence imaging results showing the distinction between positive and negative samples; and FIG. 1E depicts the surface-enhanced Raman scattering (SERS) spectra of positive and negative samples, with a significant Raman peak observed in positive samples and a flat spectrum in negative samples;

FIGS. 2A-2G depict the characterization and optimization of the CFSERS probes, in which: FIG. 2A shows the flowchart of fabrication of the CFSERS probes; FIG. 2B shows the UV-visible absorbance spectrum of the probes; FIG. 2C depicts the fluorescence spectrum of the probes; FIG. 2D shows the Raman spectrum of the probes; and FIGS. 2E-2G show the absorbance (FIG. 2E), fluorescence (FIG. 2F), and Raman intensity (FIG. 2G) correspond to FIGS. 2B-2D;

FIGS. 3A-3J depict the calibration curve establishment for CRP detection using the multimodal LFA system, in which: FIG. 3A shows the photograph of the multimodal LFA strip with CRP standards at concentrations of 2000, 1000, 500, 250, 125, 62.5, 31.25, 15.625, 7.8125, and 0 ng/mL; FIG. 3B depicts the representative fluorescence images of the multimodal LFA testing CRP standards; FIG. 3C and FIG. 3D depict the average Raman spectra from the test and control lines of the multimodal LFA for CRP standards, respectively; FIGS. 3E-3G depict that the fluorescence intensity ratios, SERS intensity ratios, and corresponding response values for the CRP standards are calculated for both the test and control lines, as shown in FIGS. 3H-3J show the calibration curves for CRP standards determined by fluorescence (FIG. 3H), SERS (FIG. 3I), and the multimodal mapping algorithm determined calibration curves for CRP standards (FIG. 3J);

FIGS. 4A-4C depict the clinical testing of urinary CRP levels using the multimodal LFA system, in which: FIG. 4A shows that urinary CRP levels, measured in 31 urine samples from healthy donors and patients with cardiovascular disease, are quantified using the multimodal LFA system; FIG. 4B depicts the correlation analysis comparing the urinary CRP results from the multimodal LFA system and ELISA for 31 clinical samples; and FIG. 4C shows the Bland-Altman analysis comparing the urinary CRP results obtained from the multimodal LFA system and ELISA for 31 clinical samples;

FIGS. 5A-5B depict the multimodal mapping algorithm for the integration of optical signal, in which: FIG. 5A is a comprehensive flowchart that illustrates the qualitative analysis employing the colorimetric signal and the quantitative analysis utilizing fluorescence and SERS signals; and FIG. 5B depicts the flowchart that outlines the procedures for standard curve establishment and clinical sample testing employing the multimodal mapping algorithm;

FIGS. 6A-6C depict the linear calibration curve of CRP standards as calculated using fluorescence (FIG. 6A), SERS modes (FIG. 6B), and a multimodal mapping algorithm (FIG. 6C);

FIGS. 7A-7C depict the human CRP standards detected by a commercial ELISA kit, in which: FIG. 7A displays the representative images of the solution products of CRP standards; FIG. 7B shows the calibration curve of the human CRP standards (1000, 500, 250, 125, 62.5, 31.3, 15.6, and 0 μg/mL); and FIG. 7C demonstrates the linear ranges of the calibration curve from 250 μg/mL to 15.6 pg/mL;

FIG. 8 depicts the comparison of the quantitative results of clinical samples detected by the multimodal LFA system and the standard plate reader;

FIGS. 9A-9B depict a significant elevation in urinary CRP levels observed in post-MI patients compared with healthy donors, in which: FIG. 9A shows the boxplot illustrating the correlation between urine CRP levels from healthy donors (59.09±27.25 ng/ml) and CVD patients (136.09±44.83 ng/mL); and FIG. 9B shows the corresponding ROC analysis;

FIG. 10 depicts the urinary CRP levels of 31 urine samples detected by ELISA from healthy donors (n=6) and CVD patients (n=25); and

FIGS. 11A-11B depict the patient stratification of healthy patients based on CRP levels in urine samples detected by ELISA, in which: FIG. 11A depicts the boxplot illustrating the correlation between urine CRP levels from healthy donors (63.69±22.14 ng/ml) and CVD patients (133.61±57.89 ng/mL); and FIG. 11B shows the corresponding ROC analysis; and

FIG. 12 depicts the raspberry-like morphology of EC@AuNP^{pNTP} hybrid nanostructures.

### DETAILED DESCRIPTION

The following description presents systems and methods for non-invasive monitoring and quantification of biomarkers in biological samples, as well as similar applications, which are provided as preferred examples. It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

Microfluidics has gained substantial prominence in personalized medicine, point-of-care (POC) diagnostics, and microfabrication. Although polydimethylsiloxane (PDMS)-based microfluidic biochips have been widely used, their intrinsic hydrophobicity limits performance in POC applications. In contrast, paper-based microfluidics—particularly LFAs—have emerged as powerful diagnostic platforms due to the natural capillary-driven fluid transport enabled by the hydrophilic properties of paper. In a typical LFA workflow, the sample is deposited onto the sample pad and subsequently wicks across the strip via capillary forces. Upon reaching the conjugate pad, labeled detection receptors are released and interact with the target analytes. The mixture then migrates to the test line, where captured receptors selectively bind the labeled analyte complex, producing a detectable signal. The sample continues to the control line, which verifies successful assay operation, before finally reaching the absorbent pad that provides sufficient capacity to maintain continuous flow. However, conventional AuNP-based LFAs often exhibit limited sensitivity and a narrow dynamic range, which constrains their ability to deliver accurate quantitative results.

Driven by these limitations, advanced optical LFAs have been developed. Fluorescence-based LFAs have achieved significant performance improvements through the use of engineered nanomaterials such as quantum dots, europium-chelate-doped polystyrene nanoparticles (ECNPs), upconverting nanoparticles, aggregation-induced emission luminogens (AIEgens), and metal-enhanced fluorescence (MEF) probes. Additionally, metal-organic frameworks (MOFs) have been incorporated into LFAs to improve sensitivity through enhanced loading capacity, signal amplification, and structural tunability. Surface-enhanced Raman scattering (SERS)-based LFAs further exploit metal nanoparticles—including AuNPs and AgNPs—to generate strong Raman enhancement, while core—shell structures such as Au@Ag and Ag@Au nanoparticles can produce even greater SERS amplification.

Among the various sensing approaches, optical LFAs remain the most widely implemented and continue to undergo rapid innovation. The present invention advances this field by introducing a novel triple-modal LFA system capable of simultaneously generating colorimetric, fluorescence, and SERS signals. Comparative analysis with existing diagnostic methods highlights several distinctive advantages of the disclosed multimodal LFA platform:
(i) it employs three fully independent detection modalities—colorimetric, fluorescence, and SERS—greatly expanding its utility across diverse analytical contexts;
(ii) ECNPs are used as the structural core of the nanocomposite probes, enabling fluorescence emission (excitation at 365 nm, emission at 610 nm) without interfering with SERS measurements obtained using a 785 nm laser, thereby preserving the independence of the optical modes; and
(iii) a multimodal mapping algorithm is developed to integrate fluorescence and SERS intensity ratios, achieving highly sensitive quantitative readout with a detection limit of 6.31 ng/ml and a broad linear dynamic range of 23.13-2000 ng/ml.

The synergistic combination of the EC@AuNP^{pNTP} composite probes and the multimodal mapping algorithm demonstrates exceptional capability for multimodal signal integration and quantitative biomarker analysis. Further improvements in detection sensitivity may be achieved by employing nanoparticles with an Ag core and a thin Au shell to enhance SERS performance.

As used herein, the term “test line” refers to a region on the nitrocellulose membrane where capture antibodies specific to the target biomarker (e.g., CRP) are immobilized. Its purpose is to capture the biomarker-probe complexes and generate a signal proportional to biomarker concentration.

In one embodiment of the present invention, the triple-mode probes (EC@AuNP^{pNTP} with antibody) bind to the target biomarker in the sample. These complexes migrate to the test line. The immobilized capture antibody binds to the biomarker-probe complex, producing colorimetric, fluorescence, and SERS signals.

Therefore, for the interpretation of the results:
Positive sample: Test line appears (color+fluorescence+SERS); and
Negative sample: Test line absent (no target to bind).

The intensity of the test-line signal reflects the amount of the biomarker present.

As used herein, the term “control line” refers to a region on the NC membrane where secondary antibodies are immobilized that bind directly to the probe (not the biomarker). As the sample migrates, excess triple-mode probes that did not bind to the biomarker continue flowing. These probes bind to the secondary antibody at the control line. This produces a universal positive signal, regardless of biomarker concentration, indicating that the strip is functioning properly and the sample has flowed correctly through the membrane. Therefore, the control line must always appear, even when the biomarker concentration is zero.

According to a first aspect of the present invention, a multimodal LFA system is provided for the non-invasive monitoring and quantification of biomarkers in biological samples. The system incorporates a specially constructed multimodal LFA stripe that integrates three signal-generation modalities—colorimetric, fluorescence, and SERS—into a single platform, thereby enabling both rapid visual detection and highly sensitive quantitative analysis.

The multimodal LFA stripe features a sample-loading region designed to accommodate a biological sample containing a target biomarker of interest. Immediately downstream of the sample-loading region is a conjugate region that stores and releases triple-mode probes upon contact with the migrating biological sample. As the sample proceeds further along the strip, it encounters a membrane zone. This membrane zone is immobilized with two types of antibodies: (i) a capture antibody that binds complexes formed between the biomarker and the triple-mode probes to generate a test line, and (ii) a secondary antibody that binds excess triple-mode probes to generate a control line. The triple-mode probes are formulated such that they inherently produce a colorimetric signal visible to the naked eye upon accumulation at the test and control lines.

The multimodal LFA system further includes a laser-emitting module configured to illuminate the membrane zone. Upon laser excitation, the triple-mode probes generate a SERS signal and a fluorescence signal in addition to the colorimetric signal. One or more optical detection devices, such as a Raman detector and a fluorescence imaging sensor, are incorporated to detect and record these multimodal optical signals.

A processor is integrated into the system to analyze the multimodal optical data. The processor is programmed to execute a multimodal mapping algorithm capable of receiving both the SERS signal and the fluorescence signal and converting them into an output value from which the concentration of the biomarker is quantitatively determined.

The triple-mode probes used in the conjugate region include several key components. Each probe contains a metal-chelate polymer nanoparticle bearing carboxyl groups on its surface and configured to emit a fluorescence signal. The probes further include a gold nanoparticle that is surface-functionalized with binders and the Raman reporter pNTP. The gold nanoparticle provides both the colorimetric signal and the SERS signal when excited by the laser. Additionally, each probe includes an antibody capable of binding to the target biomarker present in the biological sample, while also interacting with the secondary antibody on the membrane zone. In certain embodiments, the binders on the gold nanoparticle are PEG linkers containing thiol and amino groups, which can bind to the carboxyl groups of the metal-chelate polymer nanoparticle, thereby coupling the two nanoparticle components. The antibody is also connected to the metal-chelate polymer nanoparticle through these carboxyl groups. In one representative embodiment, the metal-chelate polymer nanoparticle is a europium-chelate-doped polystyrene nanoparticle, which provides a long-lifetime fluorescence signal with a significant Stokes shift and minimal interference with Raman measurements.

The multimodal mapping executed by the processor involves multiple computational steps. First, fluorescence and SERS intensity ratios are extracted from both the test line and the control line on the membrane. These ratios are then processed using a predetermined multimodal mapping coefficient that converts the fluorescence and SERS ratios into a unified response value. The unified response value is further calibrated by subtracting the response observed from a blank control sample, thereby allowing for precise compensation of background effects. The calibrated value is then correlated with a pre-established calibration curve, which maps standard biomarker concentrations to calibrated response values, enabling the precise determination of the biomarker concentration in the biological sample. In certain embodiments, the multimodal mapping coefficient is derived from standard samples by correlating fluorescence intensity ratios (R_{F}) and SERS intensity ratios (R_{R}) with known biomarker concentrations.

To ensure accurate SERS measurement, the processor may also regulate parameters of the laser-emitting module. These include optimizing exposure duration, adjusting laser intensity, and controlling the exact spatial positioning of the laser relative to the test and control lines. The system may further incorporate a colorimetric imaging module, which may be integrated into a portable device such as a smartphone or tablet, enabling the processor to receive and analyze colorimetric data in addition to fluorescence and SERS data.

In certain embodiments, the multimodal LFA stripe additionally includes an absorbent region positioned downstream of the membrane zone to collect excess biological sample. A backing layer is also included to provide mechanical stability and structural integration to the entire LFA stripe, ensuring consistent sample flow and reliable test performance.

Through the combination of triple-mode probes, multimodal signal acquisition, and advanced multimodal mapping, the disclosed LFA system allows for sensitive, quantitative, and non-invasive measurement of biomarkers across a wide dynamic range.

In accordance with a second aspect of the present invention, a method for detecting or quantifying a biomarker in a biological sample using the aforementioned multimodal LFA system is provided. In the process, a biological sample is first applied directly onto the sample-loading region of the multimodal LFA stripe. Upon application, the sample migrates laterally along the length of the stripe by capillary action. As it flows through the conjugate region, the sample encounters the triple-mode probes stored within that region. These triple-mode probes immediately interact with and bind to the biomarker present in the sample, forming complexes between the probe and the biomarker. During this migration and binding process, the triple-mode probes inherently produce multiple optical outputs, including a visible colorimetric signal, a fluorescence signal, and a Raman scattering signal.

As the sample continues to wick across the membrane zone, the complexes formed between the triple-mode probes and the biomarker are captured at the test line, where a capture antibody is immobilized. Excess triple-mode probes that do not bind to the biomarker are subsequently captured at the control line by a secondary antibody. This dual-line capture ensures the generation of distinct optical signatures at both the test and control lines, simultaneously verifying correct flow and reagent activity within the strip.

Once these signals are formed, multimodal optical signals—including the colorimetric, fluorescence, and SERS outputs—are acquired from both the test line and the control line using one or more optical detection devices associated with the multimodal LFA system. These devices may include a fluorescence imaging module, a Raman spectrometer, or a camera suitable for capturing the colorimetric signal.

After the multimodal optical data are collected, the processor analyzes these data to generate a quantitative measurement of the biomarker concentration. The processor applies a multimodal mapping approach that integrates the different signal modalities. This mapping strategy combines information from colorimetric, fluorescence, and SERS signals, transforming them into a unified analytical value. In some embodiments, the processor calculates ratios of the test-line signal to the control-line signal to reduce variability and generate a more robust representation of the biomarker level. These ratios are then processed through the multimodal mapping algorithm, which normalizes and correlates the multispectral signal inputs to produce a quantitative measurement.

In various embodiments, the biological sample used in the method may include urine, saliva, tears, blood, serum, plasma, or cell culture media. The technique is therefore compatible with a wide range of biological matrices relevant to clinical diagnostics, point-of-care testing, and laboratory research.

In some embodiments, once the biomarker concentration has been calculated, the processor communicates the result to a display interface. The concentration may be shown to a user on a smartphone, tablet, or portable optical reader that is operatively connected to the processor. This feature enables the immediate, user-friendly interpretation of results across a range of fields or clinical environments.

The method also encompasses flexibility in signal acquisition. In certain embodiments, the fluorescence, colorimetric, and Raman signals may be acquired sequentially in a defined order. In other embodiments, these signals may be acquired simultaneously using an integrated detection system capable of capturing multimodal optical outputs in parallel.

To enhance the reliability of biomarker quantification, additional processing steps may be implemented. For example, the fluorescence and Raman intensity ratios obtained from the test and control lines may be normalized using the corresponding control-line signals. This normalization step compensates for strip-to-strip variability, differences in flow rate, minor variations in reagent deposition, and other factors that could introduce measurement discrepancies. Through this combination of multimodal signal acquisition, normalization, and computational mapping, the method provides precise, robust, and non-invasive quantification of biomarkers in diverse biological samples.

### EXAMPLES

### Example 1. Development of a Triple-Modality Approach for Enhanced Biomarker Detection in Human Biofluids

LFAs are widely adopted commercial diagnostic tools for biomarker detection in human biofluids due to their simple operation and suitability for point-of-care use. However, traditional LFAs often suffer from insufficient sensitivity and generally provide qualitative or semi-quantitative results. To address these limitations, the present invention provides a multimodal LFA system that integrates three complementary optical detection modalities-colorimetric, fluorescence, and SERS-into a single platform. By combining these modalities, the system substantially expands the analytical versatility of LFAs, enabling both rapid visual screening and highly sensitive quantitative detection across a broad range of target concentrations.

The multimodal LFA system includes five distinct functional regions, each performing a specific role within the assay. These include: a sample-loading region for introducing the biofluid under test; conjugate areas that retain the CFSERS (colorimetric, fluorescence, and SERS) probes; a nitrocellulose (NC) membrane zone that immobilizes the capture anti-CRP antibody and the secondary antibody; an absorbent region for collecting waste solution; and a backing layer that provides mechanical support and integration for all functional components.

During operation, biofluid samples are applied to the sample-loading region using micropipettes, after which the sample wicks laterally across the membrane toward the absorbent region. Rapid visual inspection enables the immediate determination of strip validity. The absence of a colorimetric signal at the control line indicates assay failure, while the presence of the control line confirms proper strip function. For quantitative analysis of biomarker concentrations, a benchtop fluorescence imaging system and a Raman spectrometer equipped with a laser (i.e., 785-nm laser) are used to acquire fluorescence and SERS signals, respectively.

Raman spectroscopy is employed as a sensitive analytical technique for characterizing both liquid samples and dried samples on the multimodal LFA strip. In certain embodiments, for liquid-phase analysis, approximately 5 μL of the sample is dispensed onto a silicon wafer and subjected to Raman measurement using an instrument equipped with a 785 nm excitation laser. To minimize interference from the intrinsic silicon peak at 528 cm⁻¹ and to capture diagnostically relevant vibrational features, spectral acquisition is restricted to the fingerprint region between 600 and 1800 cm⁻¹. The raw Raman spectra are subsequently processed in MATLAB, including wavelet-based signal denoising and baseline correction. Three discrete points corresponding to the sample region on the substrate are collected, processed, and averaged to obtain the final Raman output. For analysis of multimodal LFA test strips, the strip is placed directly onto a glass microscope slide, and the Raman laser is focused onto the locations of the test line and control line for spectral acquisition, enabling direct SERS-based detection on the device.

In one embodiment, the multimodal LFA system is assembled by first patterning the NC membrane with the secondary antibody (1 mg/mL) and the primary capture antibody (1 mg/mL) using a dispensing apparatus operating at a jetting rate of 50 mm/s. The antibody-jetted membrane is subsequently dried for 24 hours. The sample-loading regions are pretreated with a conditioning buffer composed of 10 mL PBS, 0.05 mL of 100% BSA, and 0.005 mL Tween-20, followed by drying for 48 hours. In parallel, the CFSERS probe solution is dispensed onto the conjugation region at a jetting rate of 60 mm/s and dried under ambient conditions. Thereafter, the functionalized components are assembled onto a backing substrate, with the NC membrane positioned centrally, the absorbent region affixed downstream of the NC membrane, and the conjugation region affixed upstream. The conditioned sample-loading region is attached at the front end of the backing substrate to form a fully integrated lateral flow test strip. The assembled sheet is then cut into individual test strips with a precise width of 3 mm using a cutting instrument and subsequently enclosed within protective cassettes.

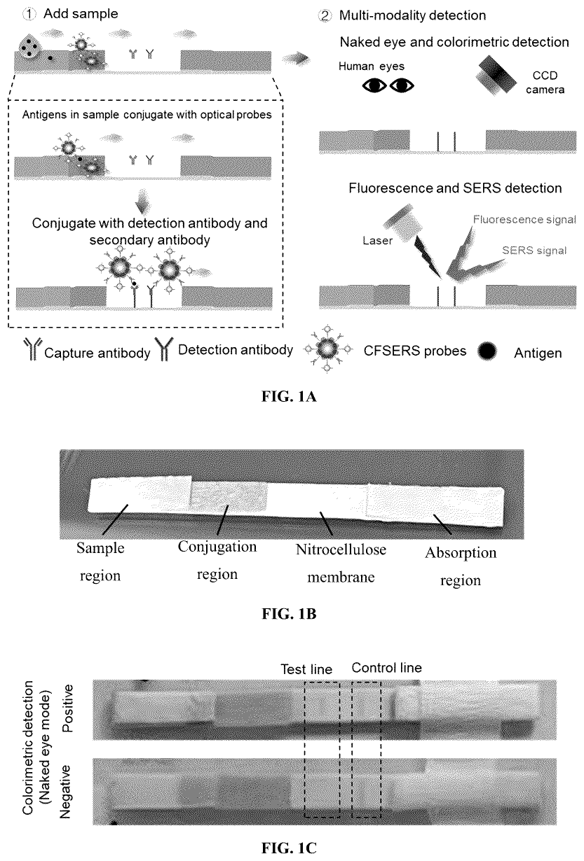

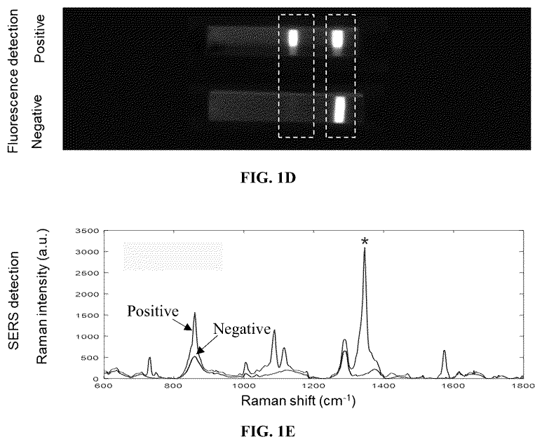

As shown in FIG. 1A, urine samples are applied to the sample pad, where the antigen (e.g., CRP) is conjugated with CFSERS probes. The nanocomposites then migrate along the lateral flow stripe, interacting with coating antibodies at the control and test lines on the nitrocellulose membrane. At the same time, the absorbent pad absorbs the excess sample.

The colorimetric mode provides an immediate naked-eye readout, while the fluorescence and SERS modes enable sensitivity and quantitative detection. The multimodal LFA system incorporates conjugation pads pre-loaded with triple-mode probes (CFSERS probes) capable of generating the three distinct optical signals (FIG. 1B). Additional structural components include a sample pad for introducing biological samples, a NC membrane containing test and control lines functionalized for CRP detection, an absorption pad for collecting excess fluid, and a backing pad that mechanically supports and integrates the complete device architecture. Together, these components form a complete full-strip multimodal LFA system.

The performance of the multimodal LFA system in differentiating positive from negative samples is evaluated using CRP standards (1 μg/mL) and blank control samples (distilled water) applied to the sample pad. In the colorimetric mode, positive samples display two visible red lines corresponding to the test and control lines. In contrast, negative samples produce only the control line (FIG. 1C). Consistent results are obtained using a fluorescence imaging reader, which readily distinguishes positive from negative samples (FIG. 1D).

In the SERS mode, positive samples exhibit pronounced Raman peaks at approximately 1346 cm⁻¹, while negative samples generate a comparatively flat SERS spectrum with no significant peak features (FIG. 1E). The apparent differences in line formation and spectral intensity across all three modalities confirm the system's capability to identify positive and negative samples accurately. Moreover, the construction of a calibration curve for CRP demonstrates the system's ability to provide sensitive and quantitative detection of biomarkers.

### Example 2. Optimization of Triple-Mode Signal Generation Using Europium Chelate-Doped Polystyrene and Gold Nanoparticle-Based Probes

To effectively integrate fluorescence and SERS detection within a single lateral flow platform, the two optical signals must be generated independently, which can be achieved by modulating the excitation wavelength. Raman signals are known to experience significant spectral interference and even complete masking when short-wavelength excitation sources are used. Accordingly, in some embodiments, Europium-chelate-doped polystyrene nanoparticles (ECNPs) are selected as the fluorescent core material due to their highly desirable photophysical properties, including an extended fluorescence lifetime and a significant Stokes shift. These characteristics render ECNPs superior to alternative fluorescent nanomaterials, such as quantum dots, for fluorescence-based LFA applications. In parallel, gold nanoparticles (AuNPs) are incorporated as metallic nanostructures because their strong surface plasmon resonance enables the simultaneous generation of colorimetric and SERS signals. As a proof of concept, pNTP is employed as the Raman reporter molecule to generate distinct and well-resolved Raman peaks. Importantly, the excitation/emission wavelengths of ECNPs (365 nm and 610 nm, respectively) are spectrally independent from the near-infrared excitation wavelength (785 nm) used to generate Raman scattering. This spectral separation ensures that fluorescence and SERS signals are produced without mutual interference.

For the preparation of AuNPs, a 10% w/v HAuCl₄ stock solution is prepared by dissolving 1 g of gold (III) chloride trihydrate (HAuCl₄·3H₂O) in 10 mL of deionized (DI) water. A 1% w/v sodium citrate solution is prepared by dissolving 0.1 g of sodium citrate tribasic dihydrate (Na₃C₆H₅O₇·₂H₂O) in 10 mL of DI water. To synthesize the nanoparticles, 100 μL of the 10% HAuCl₄ solution is introduced into 100 mL of DI water preheated to boiling. After maintaining boiling for 5 minutes, 1 mL of the 1% sodium citrate solution is rapidly added under vigorous stirring, and the mixture is heated for an additional ~10 minutes. During this process, the reaction mixture undergoes a characteristic color transition from pale yellow to blue and finally to wine red, confirming the formation of AuNPs. After the reaction is complete, the solution is cooled to room temperature, adjusted to a total volume of 100 mL, sealed, and stored at 4° C. until further use. The synthesized AuNPs exhibit an average diameter of 28.16±8.39 nm.

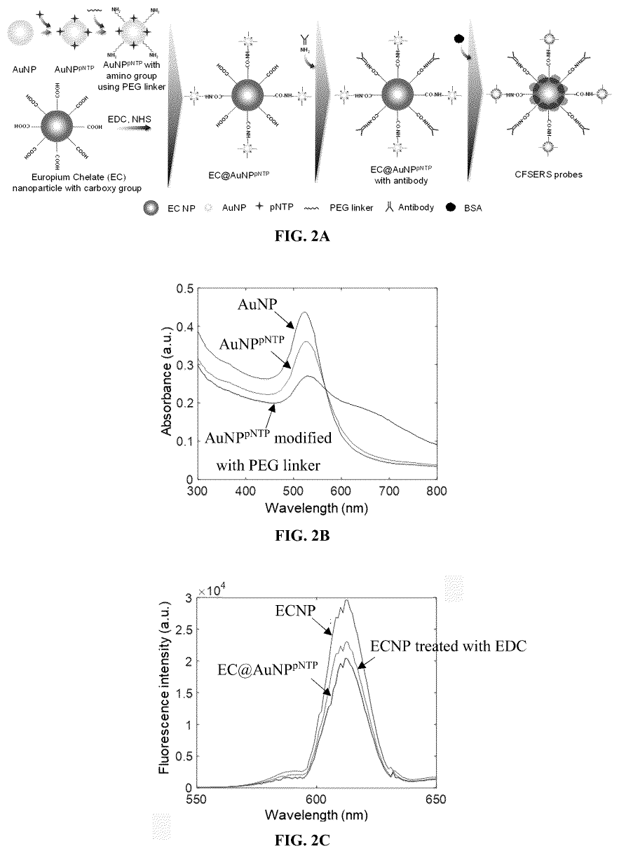

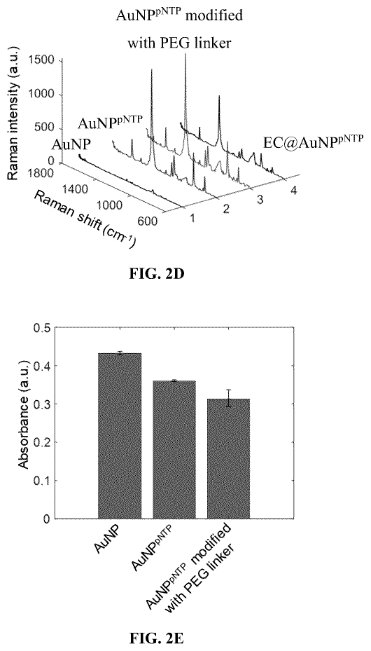

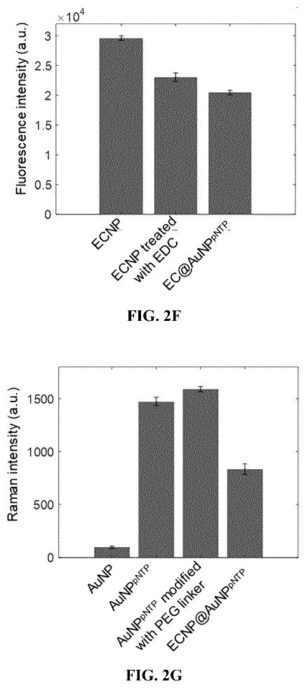

The fabrication of the CFSERS probes involves three primary stages (FIG. 2A). First, ECNPs bearing surface carboxyl groups are activated for subsequent conjugation. Second, AuNPs functionalized with pNTP are synthesized to serve as the colorimetric and SERS-active components, after which a polyethylene glycol (PEG) linker containing thiol and amino functional groups is introduced onto the AuNP surface. Third, the two nanocomponents are coupled through an amide-forming reaction between the amino groups on the PEG-modified AuNPs and the activated carboxyl groups on the ECNPs, yielding EC@AuNP^{pNTP} hybrid nanostructures exhibiting a characteristic raspberry-like morphology (FIG. 12). Following nanoparticle assembly, the CRP antibody and bovine serum albumin (BSA) are added to functionalize the composite probes for specific immunological recognition and to provide surface blocking that minimizes nonspecific interactions. The resulting CFSERS probes are stored at 4° C. until use.

Briefly, AuNP^{pNTP} probes are first prepared. A pNTP solution (10 mM, 0.5 μL) is added to an AuNP suspension (500 μL) and incubated for 2 h. The mixture is then centrifuged (6500 rpm, 10 min) to remove the supernatant. Subsequently, an HS-PEG-NH₂ solution (10 mM, 40 μL) is added to the AuNP^{pNTP} suspension (500 μL), and the mixture is gently mixed for 3 h. The resulting amino-functionalized AuNPs are centrifuged, the supernatant discarded, and the pellet washed once with DI water.

Separately, ECNPs containing surface carboxyl groups (1 mg/mL, 500 μL) are resuspended in MES buffer (10 mM, 500 μL) and washed once to activate them. EDC (2 M, 0.5 μL) and NHS (5 M, 0.5 μL) are then added to the ECNP suspension to activate the carboxyl groups, followed by a 20-min incubation. Afterward, the activated ECNPs are washed with DI water. The concentrated AuNP^{pNTP} probe solution is added dropwise to the activated ECNP suspension and gently mixed for 3 h to form AuNP^{pNTP}@ECNP composites. The resulting hybrid nanoparticles are washed with DI water. A goat anti-mouse secondary antibody solution (1 mg/mL, 20 μL) is then added to the AuNP^{pNTP}@ECNP composite suspension and mixed gently for 2 h. After a final DI-water wash, the pellet is resuspended in blocking buffer (1% BSA in PBS, 100 μL). The resulting CFSERS probes are stored at 4° C. until use.

Furthermore, to compensate for signal attenuation caused by partial probe loss during washing steps, the concentration of amino-functionalized AuNP^{pNTP} is increased two-fold (FIGS. 2E and 2G). The pronounced fluorescence intensity confirms the strong signal contribution of the ECNP core (FIG. 2F). Collectively, the characterization results obtained from absorbance measurements, fluorescence spectroscopy, and Raman spectroscopy verify the successful synthesis of AuNP^{pNTP} and its subsequent conjugation to ECNPs. The resulting EC@AuNP^{pNTP} nanocomposites are shown to reliably produce distinct colorimetric, fluorescence, and SERS outputs, demonstrating their suitability as triple-mode probes for multimodal LFA readout.

### Example 3. Multimodal Signal Integration and Quantitative Analysis Of Biomarker Concentrations

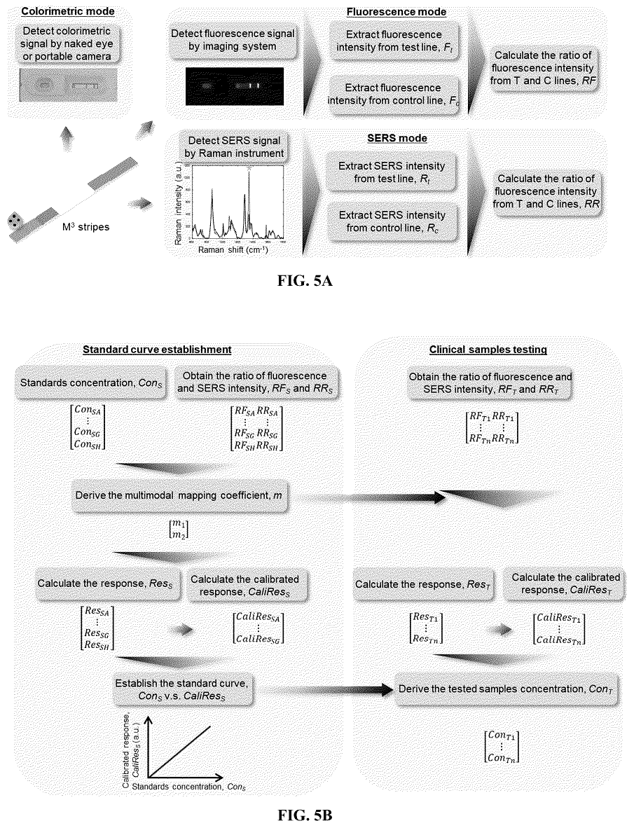

Upon application of the samples, the multimodal LFA test strip produces three distinct optical readouts: colorimetric, fluorescence, and SERS. The colorimetric signals are easily visible to the naked eye and may be rapidly acquired using any portable device equipped with a camera. For quantitative biomarker detection, a fluorescence imaging system and a Raman spectrometer are used to measure the corresponding fluorescence and SERS signals (FIG. 5A). Specifically, the optical signal, encompassing fluorescence and SERS intensity derived from the test and control lines, is meticulously extracted, and the resulting intensity ratio is derived to facilitate subsequent analysis. The intensities at the test line and control line (Fₜ, F_{c} for fluorescence; Rₜ, R_{c} for SERS) are extracted and analyzed. Optical intensity ratios, defined as R_{F}=Fₜ/F_{c} and R_{R}=Rₜ/R_{c}, are subsequently calculated. It is observed that using these test-to-control ratios yields markedly improved analytical sensitivity and quantification accuracy compared to relying solely on absolute test-line intensities.

Conventional approaches for biomarker quantification typically rely on a single detection modality and do not leverage the complementary information embedded across different optical domains. Such single-mode analysis may overlook correlations between modalities and fail to utilize the diagnostic advantages of multimodal data fully. To overcome these limitations, the present invention provides a multimodal mapping algorithm that integrates fluorescence and SERS signals to enhance calibration performance, including improvements in detection limit and linear dynamic range. Specifically, a least-squares fitting procedure is used to derive a mapping coefficient vector, m (m=[258.68,315.49]), which relates the fluorescence and SERS intensity ratios of standard samples (RFs and RR_{S}) to their known concentrations (Con_{S}) (FIG. 5B). Briefly, after obtaining the fluorescence and SERS intensity ratios, a multimodal mapping coefficient m is derived using the standard concentrations and their corresponding optical signals. This coefficient is then applied to compute an integrated response value, which is subsequently calibrated by subtracting the response obtained from the blank control. A calibration curve is generated by correlating the standard concentrations with their calibrated response values. For clinical sample analysis, response values are similarly calculated using the coefficient and the measured optical ratios. These response values are then calibrated and, using the established calibration curve, the biomarker concentrations of the tested samples are accurately determined.

The mapping coefficient represents the optimized weighting assigned to each modality. These coefficients are applied to transform the multimodal optical measurements into integrated response values (Resₛ). Background signals from blank samples (PBS) are subtracted to yield calibrated response values (CaliResₛ), which are then used to construct the calibration curve.

For quantification of clinical or unknown samples, the optical intensity ratios RF_{T} and RR_{T} are first calculated. Using the previously established mapping coefficient, these ratios are converted into integrated response values (Res_{T}). These values are then background-corrected to obtain calibrated response values (CaliRes_{T}). Finally, by applying the calibration curve to CaliRes_{T}, the biomarker concentration of the test sample is determined with high accuracy.

### Example 4. Calibration and Performance Evaluation of the Multimodal LFA System for CRP Detection

To calibrate the multimodal LFA system, eight standard CRP solutions spanning a concentration range from 2000 ng/ml to 7.81 ng/mL in two-fold serial dilutions, including a 0 ng/mL blank control, are prepared to cover the clinically relevant dynamic range of CRP. Each standard sample is analyzed using the multimodal LFA platform, which integrates colorimetric, fluorescence, and SERS modalities. Optical readouts are collected by capturing colorimetric signals with a smartphone, fluorescence signals with an imaging system, and SERS spectra with a Raman spectrometer.

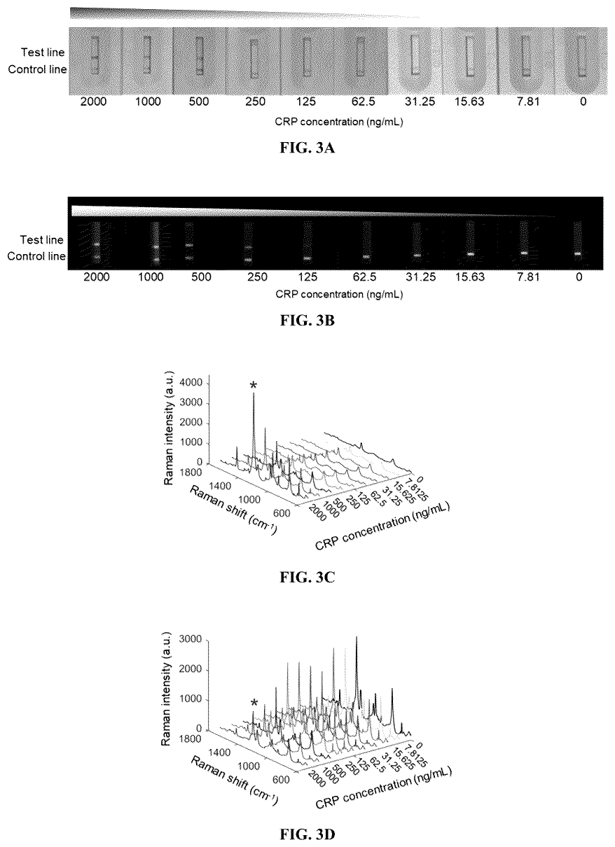

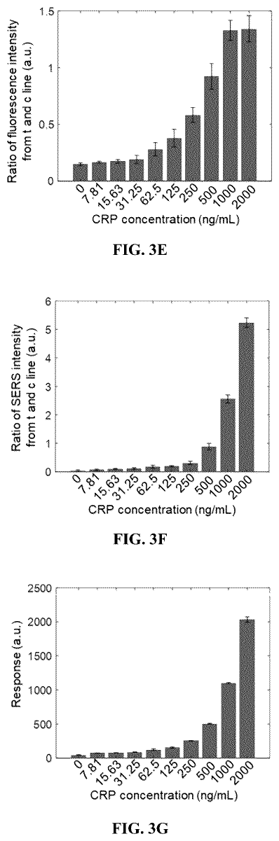

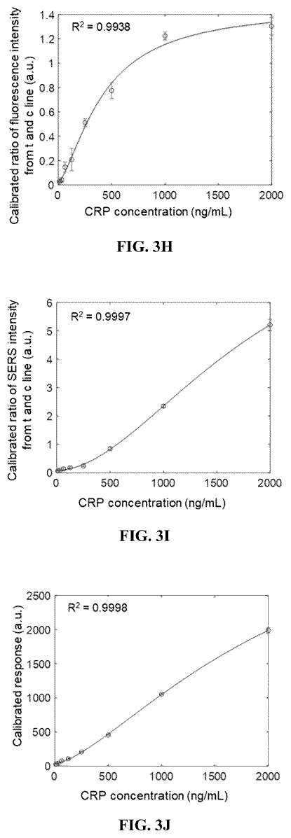

In the colorimetric mode, distinct red control lines appear on all standard test strips, confirming valid fluid flow (FIG. 3A). As the CRP concentration decreases, the test-line color visibly fades, with a naked-eye detection limit of 125 ng/mL, which is insufficient to meet clinical sensitivity requirements. By contrast, the fluorescence mode exhibits superior sensitivity, clearly resolving lower CRP concentrations (FIG. 3B). In the SERS mode, characteristic Raman peaks at approximately 1346 cm⁻¹ are detected across the CRP standards, enabling quantitative discrimination (FIG. 3C and FIG. 3D). As expected, fluorescence and SERS signal intensities decrease proportionally with declining CRP concentration, demonstrating their utility for quantitative analysis beyond the capabilities of traditional colorimetric LFAs.

To reduce background variability and evaluate system performance, the fluorescence and SERS intensities at both test (Fₜ and Rₜ) and control (F_{c} and R_{c}) lines are processed to generate ratio values (R_{F}=Fₜ/F_{c} and R_{R}=Rₜ/R_{c}). These ratios, reflecting the fluorescence (RF_{SA} to RF_{SH}) and SERS (RR_{SA} to RR_{SH}) signals across the standard concentration set, are used to construct correlation plots (FIG. 3E and FIG. 3F). The ratio values, together with the predetermined mapping coefficient m, are subsequently used to calculate integrated multimodal response signals (Res_{SA} to Res_{SH}) (FIG. 3G). The results show a monotonic increase in both ratio values (RF and RR) and integrated response values (Res) with rising CRP concentrations (Con), confirming that the multimodal mapping algorithm successfully fuses the two optical modalities into a unified quantifiable metric.

To establish the calibration curve for the fluorescence and SERS modes, the ratio of fluorescence and SERS intensities, along with the response values, is calibrated (CaliRes_{SA} to CaliRes_{SG}) by subtracting the values obtained from blank samples (PBS) (FIGS. 3H-3J). The resulting CRP calibration curves are then utilized to determine the CRP concentrations in patient urine samples. The regression formulas for the fluorescence mode and response values are derived using the best-fitting model, ensuring accurate quantification of CRP levels:

$$y = \frac{B - A}{1 + {( \frac{x}{c} )}^{D}} + A .$$

Where x denotes CRP concentration, y is the observed response, and A, B, C, and D represent the minimum response, maximum response, inflection-point concentration, and slope factor, respectively. Parameter values are determined as follows: Fluorescence mode: A=1.46, B=1.50, C=420.32, D=0.03; SERS mode: A=9.76, B=1.88, C=1876.65, D=0.08; and Integrated response: A=4159.65, B=1.47, C=2151.67, D=36.82. The correlation coefficients (R²=0.9938, 0.9997, and 0.9998, respectively) demonstrate excellent curve-fitting accuracy approaching unity.

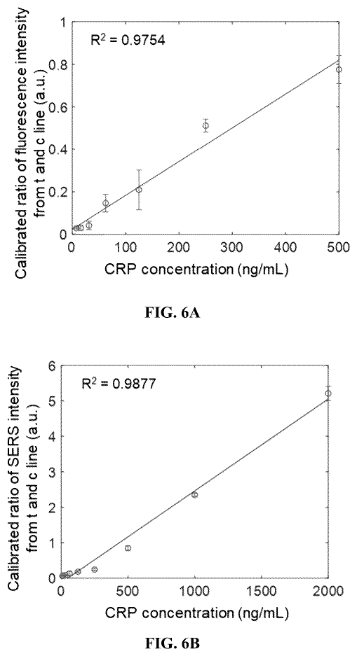

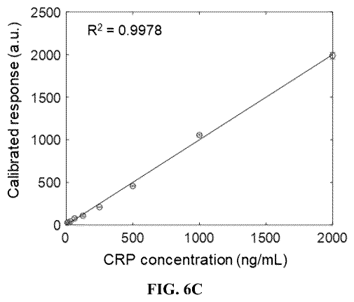

Linear regression analysis is performed to determine the linear dynamic ranges and detection limits. The linear calibration equations are obtained as: Fluorescence (0-500 ng/ml): y_{CaliRF}=0.0016x+0.0245 (FIG. 6A); SERS (0-2000 ng/ml): y_{CaliRR}=0.0026x−0.1274 (FIG. 6B); and Integrated response (0-2000 ng/ml): y_{CaliRes}=0.9988x+0.8944 (FIG. 6C). Here, x denotes CRP concentration, y denotes calibrated response, and R² represents the coefficient of determination. The corresponding R² values (0.9754, 0.9877, and 0.9978) confirm strong linearity, with the integrated approach providing the best fit.

The limits of detection (LOD) for fluorescence, SERS, and integrated modes are calculated as 9.81 ng/ml, 77.15 ng/ml, and 6.31 ng/ml, respectively. The limits of quantification (LOQ) are 68.74 ng/ml, 142.22 ng/ml, and 23.13 ng/ml, respectively. Consequently, the linear dynamic ranges are determined as:
Fluorescence: 68.74-500 ng/ml;
SERS: 142.22-2000 ng/mL; and
Integrated: 23.13-2000 ng/mL.

The integrated multimodal approach yields both the broadest linear dynamic range and the highest correlation coefficient, demonstrating its superior analytical performance. This expanded dynamic range is particularly advantageous when testing unknown clinical samples, reducing the need for dilution or other sample-preprocessing steps.

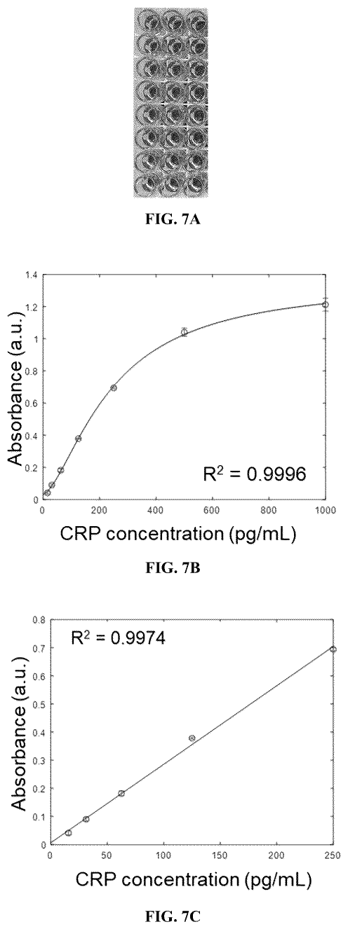

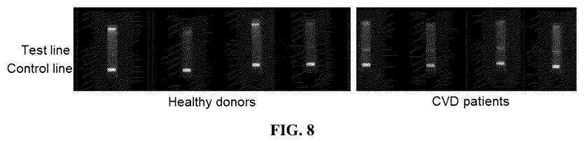

The analytical performance of the multimodal LFA system is further compared with that of a commercial ELISA kit. The ELISA exhibits an LOD of 3.39 pg/mL, LOQ of 17.04 μg/mL, and a narrow linear dynamic range of 17.04-250 pg/mL (FIGS. 7A-7C). However, its restricted dynamic range, requirement for extensive preprocessing, multiple washing steps, and long assay time (>12 h) make ELISA unsuitable for point-of-care (POC) applications, unlike the rapid and wide-range multimodal LFA described herein.

### Example 5. Clinical Validation of the Multimodal LFA System for Urinary CRP Detection in Healthy Donors and Patients

Urine samples (n=6) from healthy donors are obtained from Innovative Research, and an additional 25 urine samples are collected from patients diagnosed with cardiovascular diseases (CVDs). Ethical approval for this study is obtained from the Joint Chinese University of Hong Kong-New Territories East Cluster Clinical Research Ethics Committee (Ref. No. 2021.457). Patient urine samples are collected using sterile plastic collection cups, transferred to the laboratory, aliquoted into 2 mL tubes, and stored at −80° C. until analysis. Before testing, frozen urine samples are thawed at room temperature. For performance evaluation, CRP concentrations measured by the multimodal LFA system are compared against gold-standard measurements obtained using a commercial plate reader.

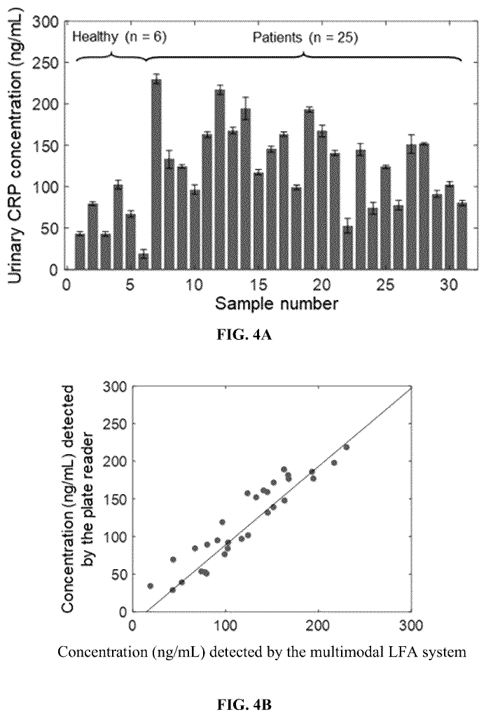

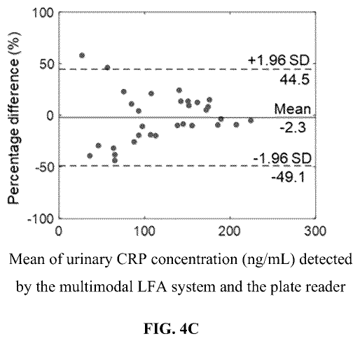

The multimodal LFA system demonstrates excellent analytical performance for urinary CRP detection and is well-suited for point-of-care clinical applications. To assess urinary CRP levels, the system is applied to urine samples from both healthy donors (n=6) and patients with CVD (n=25). Fluorescence images are acquired using a benchtop imaging system (FIG. 8), followed by SERS measurements using a Raman spectrometer. Fluorescence intensity ratios (RF_{T}) and SERS intensity ratios (RR_{T}) between the test and control lines are calculated. These ratios are subsequently converted into integrated response values (Res_{T}) using the previously derived multimodal mapping coefficients. After calibration (yielding CaliRes_{T}), urinary CRP concentrations (Con_{T}) are quantified using the established standard calibration curve (FIG. 4A). Higher integrated response values observed in the patient group are consistent with elevated inflammatory status commonly associated with cardiovascular diseases.

Quantitative CRP results obtained from the multimodal LFA system are compared with measurements obtained using a commercial ELISA assay (FIG. 10). A Passing-Bablok regression analysis is performed to evaluate correlation, yielding the regression equation y=15.19+1.04x and a Spearman correlation coefficient of 0.94, indicating strong monotonic agreement between the two measurement methods (FIG. 4B). In addition, Lin's concordance correlation coefficient is determined to be 0.9377, further confirming the high concordance between multimodal LFA measurements and ELISA results.

A Bland-Altman analysis is conducted to assess agreement and identify potential bias between measurements from the multimodal LFA system and gold-standard plate reader results (FIG. 4C). The analysis evaluates whether differences fall within the acceptable limits of agreement (δ+1.96 s), where & represents the mean difference and s denotes the standard deviation of differences. The results show that 93.55% of data points fall within these limits. A mean bias of 2.3% is observed, with 95% confidence intervals ranging from −49.1% to +44.5%, suggesting that the discrepancy between methods slightly increases at higher urinary CRP concentrations.

Taken together, Lin's concordance correlation coefficient and Bland-Altman analyses demonstrate a strong correlation (0.9377) and high agreement (93.55%) between the multimodal LFA system and standard ELISA measurements. These results confirm that the multimodal LFA system provides reliable, quantitative, and clinically relevant detection performance, supporting its potential for point-of-care monitoring of urinary biomarker levels.

### Example 6. Clinical Correlation of Urinary CRP as a Diagnostic Biomarker with the Multimodal LFA System for Point-of-Care Utility

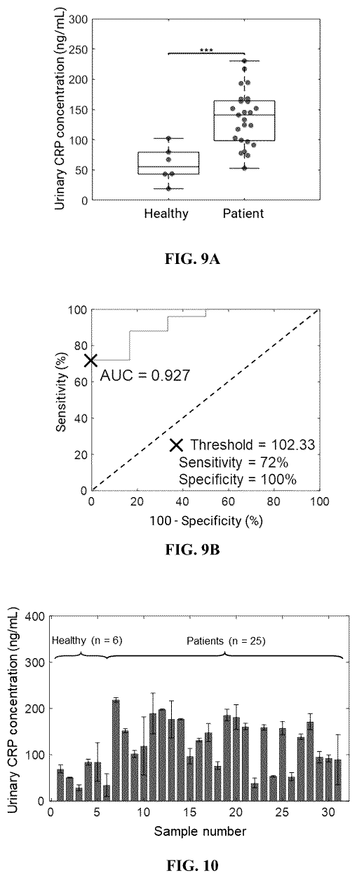

To further evaluate the clinical relevance of urinary CRP levels for distinguishing healthy donors (n=6) from post-myocardial infarction (MI) patients (n=25), urinary CRP concentrations obtained from each group are compared, and diagnostic thresholds are established using receiver operating characteristic (ROC) analysis. A significant elevation in urinary CRP levels is observed in post-MI patients compared with healthy donors (p<0.001), indicating an increased inflammatory burden associated with myocardial infarction (FIG. 9A).

ROC analysis is performed to determine optimal classification thresholds for separating healthy donors from post-MI patients (FIG. 9B). The resulting area under the ROC curve (AUC) of 0.927 demonstrates excellent discriminatory performance of urinary CRP. Using Youden's index to maximize sensitivity and specificity, a threshold of 102.33 ng/ml is identified, yielding a sensitivity of 72% and a specificity of 100%. The combination of high sensitivity (72%), perfect specificity (100%), and a strong AUC (0.927) highlights the clinical potential of urinary CRP as a non-invasive biomarker for disease stratification. These findings further support the utility of the M3 LFA system as a robust point-of-care platform for non-invasive disease monitoring.

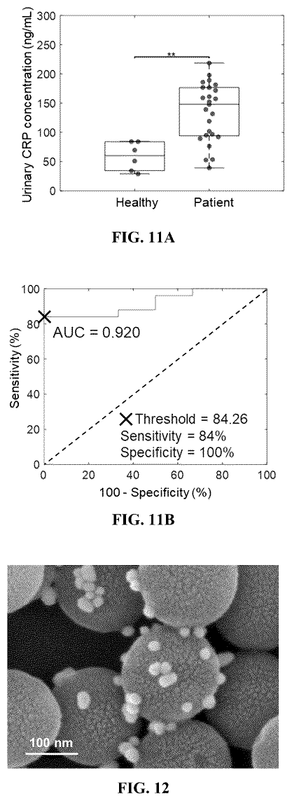

Comparable stratification is achieved using measurements obtained from a commercial plate reader (FIGS. 11A-11B). A urinary CRP threshold of 84.26 ng/ml effectively discriminates between healthy individuals and patients, yielding high sensitivity (84%) and specificity (100%). Together, these results underscore the value of the multimodal LFA system in accurately detecting urinary CRP and demonstrate its promise as a reliable tool for point-of-care clinical assessment.

In summary, the multimodal LFA system provided by the present invention integrates three complementary detection modalities—colorimetric, fluorescence, and SERS (CFSERS probes)—to enable highly sensitive, non-invasive quantification of biomarkers. To realize this capability, EC@AuNP^{pNTP} nanocomposites are engineered to generate distinct multimodal optical signals with minimal spectral interference, facilitated by the significant Stokes shift of the ECNP core. In addition, a dedicated multimodal mapping algorithm is developed to fuse fluorescence and SERS readouts, thereby enhancing analytical performance by lowering the detection limit and expanding the linear dynamic range of the CRP calibration curve. As a proof of concept for non-invasive biomarker monitoring, urinary CRP levels in clinical samples are quantified using the multimodal LFA system, yielding results that show strong agreement with those obtained from ELISA measurements. Collectively, these findings demonstrate the substantial potential of the multimodal LFA platform for non-invasive, point-of-care disease monitoring.

The electronic functional units and modules of the systems, such as the processors, in accordance with the embodiments disclosed herein may be implemented using computing devices, computer processors, or electronic circuitries including but not limited to application specific integrated circuits (ASIC), field programmable gate arrays (FPGA), graphical processing units (GPU), microcontrollers, and other programmable logic devices configured or programmed according to the teachings of the present disclosure. Computer instructions or software codes that run on computing devices, computer processors, or programmable logic devices can be readily prepared by practitioners skilled in software or electronic art, based on the teachings of the present disclosure.

All or portions of the methods in accordance with the embodiments may be executed in one or more computing devices, including server computers, personal computers, laptop computers, mobile computing devices such as smartphones, and tablet computers.

The embodiments may include computer storage media, as well as transient and non-transient memory devices, which store computer instructions or software codes that can be used to program or configure computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for illustration and description. It is not intended to be exhaustive or to limit the invention to the precise forms disclosed. Many modifications and variations will be apparent to the practitioner skilled in the art.

The embodiments were chosen and described to explain best the principles of the invention and its practical application, thereby enabling others skilled in the art to understand the invention for various embodiments and with various modifications suited to the particular use contemplated.

1. A multimodal lateral flow assay (LFA) system for non-invasively monitoring biomarkers, comprising:
a multimodal LFA stripe, comprising:
a sample-loading region configured to receive a biology sample comprising a biomarker;
a conjugate region comprising triple-mode probes; and
a membrane zone immobilized with a capture antibody configured to bind complexes of the triple-mode probes and the biomarker and form a test line, and a secondary antibody configured to bind the triple-mode probes and form a control line, wherein the triple-mode probes are operable to generate a colorimetric signal;
wherein the conjugate region is disposed adjacent to the sample-loading region and the membrane zone is disposed adjacent to the conjugate region;
a laser-emitting module configured to illuminate the membrane zone to generate a surface-enhanced Raman scattering (SERS) signal and a fluorescence signal from the triple-mode probes;
one or more optical detection devices configured to detect and receive the SERS signal and the fluorescence signal; and
a processor configured to execute a multimodal mapping for analyzing the SERS signal and the fluorescence signal to obtain a concentration of the biomarker.

2. The multimodal LFA system of claim 1, wherein the triple-mode probes comprise:
a metal-chelate polymer nanoparticle having carboxyl groups on its surface and configured to emit the fluorescence signal;
a gold nanoparticle functionalized h binders and 4-nitrobenzenethiol (pNTP) configured to produce the colorimetric signal and the SERS signal under laser excitation; and
an antibody capable of binding both the biomarker in the biological sample and the secondary antibody in the membrane zone;
wherein the binders bind to the carboxyl groups, and the antibody is coupled to the metal-chelate polymer nanoparticle via the carboxyl groups.

3. The multimodal LFA system of claim 1, wherein the multimodal mapping comprises:
extracting fluorescence and SERS intensity ratios from the test and control lines;
applying a predetermined multimodal mapping coefficient to convert the fluorescence and SERS ratios into a unified response value;
calibrating the unified response value by subtracting a blank-sample response; and
determining the biomarker concentration in the biological sample based on a calibrated curve correlating standard concentrations with calibrated response values.

4. The multimodal LFA system of claim 2, wherein the metal-chelate polymer nanoparticle comprises a europium-chelate-doped polystyrene nanoparticle.

5. The multimodal LFA system of claim 2, wherein the binder comprises a polyethylene glycol (PEG) linker comprising thiol and amino functional groups.

6. The multimodal LFA system of claim 1, further comprising an absorbent region positioned downstream of the membrane zone for absorbing excess biological sample.

7. The multimodal LFA system of claim 1, further comprising a backing layer providing mechanical support and structural integration to the multimodal LFA stripe.

8. The multimodal LFA system of claim 3, wherein the multimodal mapping coefficient is derived from standard samples by correlating fluorescence intensity ratios (R_{F}) and SERS intensity ratios (R_{R}) with known biomarker concentrations.

9. The multimodal LFA system of claim 3, wherein the processor determines the biomarker concentration by applying the calibrated response value to a calibration curve.

10. The multimodal LFA system of claim 1, wherein the processor controls exposure duration, laser intensity, and spatial positioning of the laser-emitting module to optimize SERS signal acquisition.

11. The multimodal LFA system of claim 1, wherein the colorimetric signal is acquired using a camera integrated into a smartphone, tablet, or portable imaging module operatively connected to the processor.

12. A method for detecting or quantifying a biomarker in a biological sample using the multimodal LFA system of claim 1, the method comprising:
applying a biological sample to the sample-loading region of the multimodal LFA stripe;
allowing the sample to migrate along the stripe through the conjugate region comprising the triple-mode configured to generate the complexes of the triple-mode probes and the biomarker, to create colorimetric, fluorescence, and Raman scattering signals;
capturing the complexes of the triple-mode probe at the test line of the membrane zone;
capturing excess multimodal probes at the control line of the membrane zone;
acquiring multimodal optical signals from the test line and the control line using one or more optical detection devices; and
processing the optical signals by the processor to generate a quantitative measurement of the biomarker concentration;
wherein processing the optical signals comprises applying a multimodal mapping that combines the colorimetric, fluorescence, and SERS signals.

13. The method of claim 12, wherein generating the quantitative biomarker measurement comprises computing signal ratios between the test line and the control line.

14. The method of claim 12, wherein the biological sample comprises urine, saliva, tears, blood, serum, plasma, and cell culture media.

15. The method of claim 12, wherein the biomarker concentration is displayed to a user on a smartphone, tablet, or portable optical reader operatively connected to the processor.

16. The method of claim 12, wherein the fluorescence, colorimetric, and Raman signals are acquired sequentially or simultaneously.

17. The method of claim 12, wherein the processing further comprises normalizing fluorescence and Raman intensity ratios using control-line signals to reduce strip-to-strip variability.
